(19) United States
(12) Patent Application Publication
(10) Pub. No.: US 2002/0122742 A1
(43) Pub. Date: Sep. 5, 2002

(54) Process for stripping and sterilizing the inside of a container and device for its implementation

(76) Inventors: Rene Wajsfelner, Fresnes (FR); Jacques Depoizier, Saint Julien de Beychevelle (FR); Jean-Paul Longuemard, Villebon (FR)

Correspondence Address: YOUNG & THOMPSON, 745 SOUTH 23RD STREET 2ND FLOOR, ARLINGTON, VA, 22202

(21) Appl. No.: 10/062,662

(22) Filed: Feb. 5, 2002

Related U.S. Application Data
(63) Continuation-in-part of application No. 09/720,316, filed on Mar. 1, 2001, now Pat. No. 6,368,554.
Application No. 09/720,316 is a 371 of international application No. PCT/FR99/01363, filed on Jun. 9, 1999.

(30) Foreign Application Priority Data
Jun. 26, 1998 (FR) 98 08140

(51) Int. Cl. A61L 2/00; B01J 19/08; B01J 19/12

(52) U.S. Cl. 422/22; 422/186.05; 422/907

(57) ABSTRACT

A pulsed radiation produced by an intense optical source is applied to the inner surface of a wooden container having a surface layer of organic and/or mineral deposit. Each pulse is of sufficiently short duration having sufficiently high treating energy density per unit area to bring about sublimation of the surface layer. The resulting exposed container surface is sanitized by the heat released by the radiation.

### FIELD OF THE INVENTION

The present invention relates to a process for stripping and sterilizing the internal surface of a container, for example made of wood, metal, concrete or any other material, especially a wooden cask having a surface layer of a coating of organic and/or mineral material, especially a coating of tannin resulting from the maturation of a wine in a cask, as well as to a device for its implementation.

Wooden casks will be of particular interest in the remainder of the description, but it should be well understood that the invention is in no way limited thereto and that it can be applied to any type of container, whatever it is made of.

### BACKGROUND OF THE INVENTION

During the period of maturing wines in a barrel, it is generally accepted that the wood transfers various substances, such as furans, lactones, aldehydes, phenolic acids, phenols and ketones to the wine. The barrel puts the wine in an oxidizing balance and acts as a kind of micro-doser of oxygen, which allows a first oxidation-reduction aging of the wine. It is generally assumed that a new barrel transfers tannic substances to the wine while an old barrel transfers substances from the decomposition of the wood. A one-year-old barrel, i.e. a barrel having already served to mature a wine for one year, generally gives a taste of pure wood to the wine, while a six-year-old barrel generally gives a rancid taste.

To mature certain wines, wooden barrels, the inside of which has been scorched on the surface, are also used to transfer other substances, such as phenolic compounds, furanic aldehydes and color, to the wine.

Usually a barrel may serve to mature up to four wines over a period of 4 to 6 years. After this period, the barrel can no longer serve as a maturing tool since the wine has penetrated by about 5 to 10 mm into the thickness of the barrel which has a thickness of about 22 to 27 mm, this penetration of the wine causing sealing of the wood pores by the tannin coatings and by the alterations in the compounds of the wood, such as the phenolic compounds, tartaric acid, etc., which prevents the subsequent transfers of substances between the wood and the wine, which transfers are essential to the maturation of the wine. These old barrels may still serve as storage containers, but this is not usually the case, since microbe-related accidents may arise during storage, between the wine and the coatings covering the internal surface of the barrel.

For the maturation barrels, the quality of the wood used is very important and, in French vineyards, the wood used generally comes from oaks of about 150 to 300 years of age, which therefore have a very long renewal time faced with a very greatly increasing recent demand.

In order to reduce the cost of the barrels and to save the limited national heritage in oaks, a process for renovating the barrel has already been proposed.

One solution consists in carrying out a mechanical stripping, using a plane or a sander, inside the barrel, then in possibly carrying out scorching, in order to regain the organoleptic nature which is characteristic of a new barrel. However, this solution is lengthy and expensive to implement and does not allow the barrel to be sterilized against microbial infections. Furthermore, such a mechanical stripping leads to removing an appreciable thickness, several millimeters, of the barrel, which limits the number of possible renovations.

Another solution consists in chemically cleaning the barrel, but this solution is very cumbersome to implement and expensive.

Furthermore, the current renovation processes give quite disappointing results for the quality of the wines, since stripping which is too intense leads to a “plank” taste by completely renovating the raw wood, while stripping which is too light has no effect. Furthermore, during the renovation of the barrel, it is difficult to reproduce the initial traditional scorching, since when the barrel is too scorched, it develops strange characteristics.

### SUMMARY OF THE INVENTION

The object of the invention is to propose a process for stripping and sterilizing the internal surface of a container, which is both simple to implement and which allows a very high number of renovations.

The invention is based on the principle of renovation by laser which ensures accurate and selective stripping at a controlled temperature, by photoremoval of the biological stains, for example fungal, mold, polychlorophenol and chloroanisole compounds, and/or mineral stains which are deposited over the internal surface of the container. Since the biological stains have physical characteristics which are different to those of the material forming the container, the heat increase during the absorption of the light produced by the laser will be faster in the coating of organic and/or mineral material than in the container, which makes it possible to remove the biological and/or mineral stains without causing a transfer of energy to within the material forming the cask.

For this purpose, the subject of the invention is a process for stripping and sterilizing the internal surface of a container made of wood, metal, concrete or some other material, having a surface layer of a coating of organic and/or mineral material, especially a coating of tannin resulting from the maturation of a wine in a cask, characterized in that it consists in applying, over the surface to be treated, pulsed radiation produced by an intense optical source, each pulse having a duration which is short enough and an energy density per unit area to be treated which is high enough to cause the sublimation of the said surface layer, the surface of the container thus stripped being sterilized by the heat released by the radiation. Using the invention, the layer of organic and/or mineral material is sublimed, which generates a gaseous plasma in the form of smoke, which avoids the drawbacks connected with the use of an aqueous solution.

Advantageously, each pulse has a duration of between 10 and 200 ns and an energy density of between about 1 and 9 J/cm², preferably between 6.5 and 8 J/cm², and an energy of about 2J. For example, each pulse has a duration of about 100 ns and an energy density between about 1 and 8 J/cm².

A long pulse duration, for example of the order of ms or μs, would lead to a transfer of energy into the material forming the container and a low rate of ejection of the sublimed marks, while the organic and/or mineral coatings have to be removed over a small thickness, quickly and without consuming too much energy. With a pulse duration of about 100 ns, a very high peak value is obtained for the beam, which causes a high ejection rate of the sublimed organic and/or mineral material and low diffusion of the heat into the material forming the container.

According to another characteristic, the process consists in applying, over each unit area, from 2 to 20 pulses, preferably between 2 and 10 pulses, depending on the type of material of the container to be treated, the state of the surface to be treated and the thickness of the organic and/or mineral coating.

According to another characteristic, the radiation is determined so as to cause a quasiadiabatic sublimation of the layer of organic and/or mineral material on the surface to be treated. In particular, provision can be made for 80% of the heat produced by the radiation to be absorbed by the surface layer during sublimation, the remaining 20% being dissipated within the thickness of the material forming the container.

Preferably, each pulse causes the sublimation of about 20 μm thickness of material on the surface to be treated.

Advantageously, the process consists in evacuating the gaseous plasma produced during the sublimation, by sucking it up or blowing it out using an inert gas or air.

According to another characteristic, the intense optical source is a laser source, for example a CO₂ laser source at atmospheric pressure and with transverse excitation.

Advantageously, the process further comprises the steps of starting to strip a portion of the internal surface, measuring at least one property among a calorimetric property of the portion, an acoustic property of the interaction between the portion and the pulsed radiation and a physical property of smoke generated by the stripping of the portion, and comparing measurement data corresponding to the said at least one property with predetermined target data representative of a final state of the internal surface to be obtained, and stopping stripping the portion when the measurement data substantially matches the target data.

Preferably, the step of measuring a calorimetric property of the internal surface portion comprises illuminating the portion with visible light and measuring a spectral property of the light reflected by the portion to determine a dominant color of the portion.

Advantageously, the step of measuring a physical property of smoke generated by the stripping of the portion comprises measuring a optical extinction coefficient of the smoke for at least one of an infrared wavelength, a visible wavelength and an ultraviolet wavelength.

Preferably, the step of measuring an acoustic property of the interaction between the portion and the pulsed radiation comprises measuring ultrasounds emitted by a plasma generated by the said interaction.

According to another aspect of the invention, for a wooden container, the process consists, simultaneously with or subsequent to the step of stripping and sterilizing, in applying over the surface to be treated a second intense optical radiation, the said second radiation being applied continuously or quasi-continuously for a duration which is long enough and with an energy density per unit area to be treated which is high enough to cause scorching of the wood on the surface. Advantageously, this second radiation is applied by a laser source with a defocused beam or by beam scanning.

Preferably, the second radiation has a power density of between 50 and 200 W/cm² for a duration of application of about 0.05 to 0.2 seconds. In this case, the second radiation preferably has an energy density per unit area to be treated of about 20 J/cm².

Although the energy density received by the wood, in the case of scorching or toasting, is greater than that for stripping, the total energy is transferred over a long time during scorching, which means that the heat diffuses into the wood and chars it on the surface, while, in the case of stripping, the energy is applied over a very short time, causing instant sublimation of the organic layer.

In another variant, the second radiation is applied by an infrared or ultraviolet lamp, for example, a lamp having a power of 70 W for an application time of several minutes, with a distance of a few centimeters between the radiation source and the surface to be treated.

Advantageously, the process further comprises the steps of starting to scorch a portion of the internal surface, measuring at least one property among a calorimetric property of the portion and a physical property of smoke generated by the scorching of the portion, and comparing measurement data of the said at least one property with predetermined target data representative of a final state of the internal surface to be obtained, and stopping scorching the portion when the measurement data substantially matches the target data.

The subject of the invention is also a device for implementing the aforementioned process, characterized in that it comprises an intense optical source capable of producing pulsed radiation in order to strip and sterilize the internal surface of the container, a waveguide connected to the optical output of the source, an optical focusing head connected to the output of the waveguide, in order to define the cross section of interaction with the surface to be treated and thus the energy density to deposit per unit area, a robot for the relative movement between the optical head and the internal surface of the container to be treated, and a central control unit in order to control and synthesize, on the one hand, the source parameters such as the number of pulses to be applied per unit area, the impulse frequency and the radiation power of the source, and on the other hand, the movements to be carried out by the robot in order to treat the entire internal surface of the container.

Advantageously, the robot is capable of making the said optical head pivot through an angle of about 120° with respect to the axis of the container.

According to another characteristic, the robot is capable of driving the optical head in relative rotation about the axis of the container with respect to the container.

According to yet another characteristic, the robot is capable in driving the container in relative axial translation with respect to the optical head, which may in this case be connected to a telescopic or extensible waveguide.

Preferably, the optical head is located at a distance from the surface to be treated of about a few tens of centimeters.

According to another aspect of the invention, the device comprises a camera for displaying the surface treatment, the said camera being connected to a display screen and to the central control unit in order to control the surface treatment visually and in real time.

According to another characteristic, the optical head is arranged so as to penetrate inside the container, for example by the bunghole of a cask made of wood, or by a hole specially made in one of the heading pieces of the cask or else by one of the ends of the cask from which the heading piece has been removed.

Preferably, a calorimetric sensor is coupled to the central control unit for measuring a dominant color of the internal surface, the calorimetric sensor being capable of relative orientation with respect to the internal surface.

Advantageously, in that case, the robot is capable of aiming the optical head and the calorimetric sensor at a same portion of the internal surface for measuring the dominant color of the portion between two pulses of the pulsed radiation. The central control unit is capable of comparing the dominant color of the portion measured by the calorimetric sensor with a predetermined target color to be obtained, the robot being capable of aiming the optical head and the calorimetric sensor at another portion of the internal surface in response to a substantial match between the target color and the dominant color of the portion as measured.

Advantageously, the device comprises a pipe for sucking up or blowing out smoke generated by the stripping.

The purpose of removing the gaseous plasma is to avoid, on the one hand, any recontamination of the treated surface and surroundings, on the other hand, any interference with the optical beam and with any display camera.

Preferably, a smoke analyzer is coupled to the central control unit for measuring an optical extinction coefficient of the smoke sucked up by the pipe for at least one of an infrared wavelength, a visible wavelength and an ultraviolet wavelength.

According to another characteristic, the robot is capable of moving the pipe in coordination with the optical head such as to keep an inlet portion of the pipe adjacent to a portion of the internal surface at which the optical head is aimed.

According to another characteristic, a microphone is coupled to the central control unit for measuring a sound pattern generated by the interaction between the pulsed radiation and the internal surface.

According to another characteristic, the device comprises a second intense optical source for producing the second radiation for scorching a container made of wood.

### BRIEF DESCRIPTION OF THE DRAWINGS

In order to better understand the object of the invention, several embodiments shown on the appended drawing will now be described, by way of purely illustrative and non-limiting examples.

In this drawing:

FIG. 1 is a block diagram of a first embodiment of the device of the invention adapted for a test on a sample;

FIG. 2 is a simplified partial diagram of a second embodiment of the device of the invention for stripping a cask;

FIG. 3 is an enlarged view of a detail of FIG. 1, showing the stripping region.

FIG. 4 is a schematic diagram showing a third embodiment of the device of the invention for stripping and scorching a cask;

FIG. 5 is a block diagram of a measurement system of the device of FIG. 4, and

FIG. 6 is a block diagram of a process for operating the device of FIG. 4.

### DETAILED DESCRIPTION OF THE INVENTION

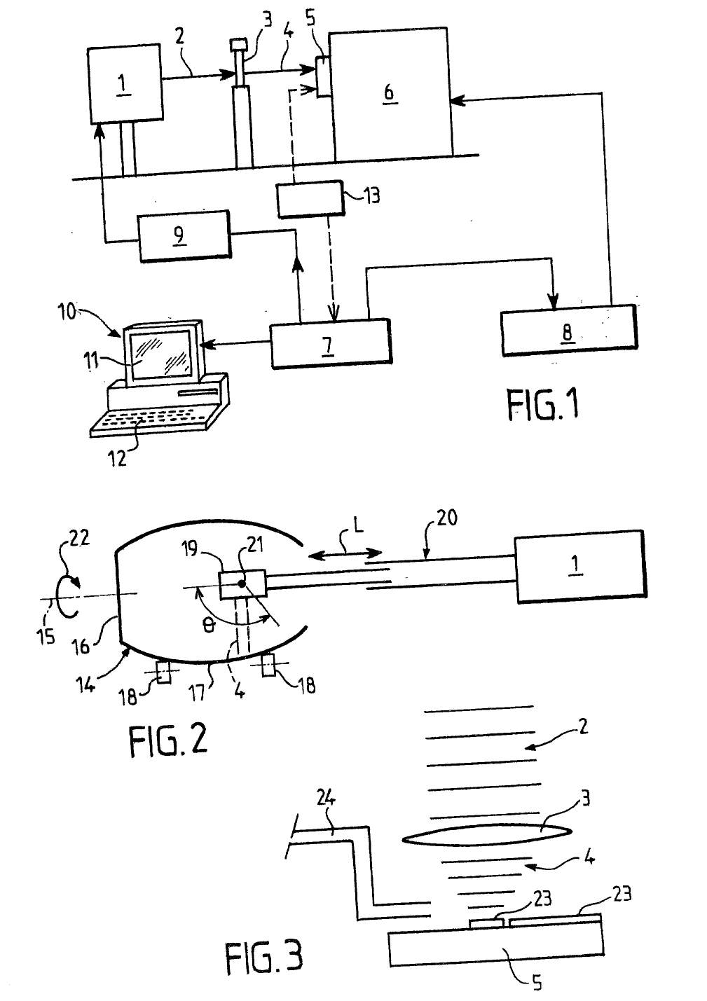

In FIG. 1, a laser source 1 is shown which is intended to produce a laser beam 2 in the direction of a convergent lens 3 which causes a beam 4 to converge on a stave 5 of a wooden cask. The stave 5 is held by a robot 6. A central control unit 7 is connected by a robot control interface 8 to the robot 6, and by a synchronization interface 9 to the laser source 1. The central control unit 7 is combined with a peripheral unit 10 formed by a screen 11 and a keyboard 12. A camera 13 may also be connected to the central control unit 7, as shown by dashed lines, in order to display the treatment of the surface of the stave 5. Although the apparatus illustrated in FIG. 1 is adapted to carry out tests on a sample 5, the general principle of the invention remains substantially the same.

FIG. 2 shows a wooden barrel 14, bulging in the middle, made in a known manner from staves which are assembled and hooped, one of the ends of which is closed by a circular heading piece 16 and the other end of which is open in the direction of the laser source 1. The barrel 14 rests by the external convex surface of its side wall 17 on rotating rollers 18 which are intended to be rotated about an axis parallel to the axis 15 of the barrel 14, by the aforementioned robot 6.

The aforementioned convergent lens 3 is included in an optical focusing head 19 which is connected to the laser source 1 by a waveguide 20 which in this case is telescopic in the axial direction indicated by the double arrow L, but which, as a variant, could be extensible. The waveguide 20 is substantially aligned with the axis 15 of the barrel 14. The optical head 19 is articulated with respect to the waveguide 20, about a horizontal axis 21 which is perpendicular to the axis 15 of the barrel. The optical head 19 is designed to pivot about this axis 21 through an angle θ of about 120° , such that the convergent beam 4 exiting from the optical head 19 is able to pivot between a position (not shown) where the said beam 4 is aligned with the axis 15 of the barrel in the direction of the heading piece 16, and a position inclined by 30° with respect to the vertical, in the direction of the open end of the barrel. Thus the optical head 19 can scan the entire internal surface of the barrel, that is the internal surface of the heading piece 16 and the internal concave surface of the side walls 17 of the barrel.

The rotating rollers 18 allow the barrel 14 to be turned about its axis 15, as shown by the arrow 22. Of course, as a variant, the waveguide 20 could be designed to rotate axially, instead of rotating the barrel 14.

FIG. 3 shows that the beam 2 coming from the optical source 1 is converted into a convergent beam 4 by the lens 3, in order to make this beam converge on a region of predetermined limited area of the internal wall of the stave 5, in order to sublimate a layer of organic material coating 23. The smoke produced by the sublimation of the layer of organic material 23 may be sucked up or blown out via a pipe 24 inserted in the barrel 14, preferably close to the region of treatment of a stave 5.

The pipe for sucking up or blowing out the smoke 24 may be combined with the camera 13. The camera 13 is, preferably, provided with an autofocus objective lens, making it possible to view the surface to be treated on the control screen 11, and therefore for the operator to control, without risk, the quality of the stripping and of the scorching.

Using the combined movements of the waveguide 20 in the axial direction of the barrel, of the axial rotation of the barrel 14 and of the limited angular pivoting of the optical head 19, the entire internal surface of the barrel can be treated.

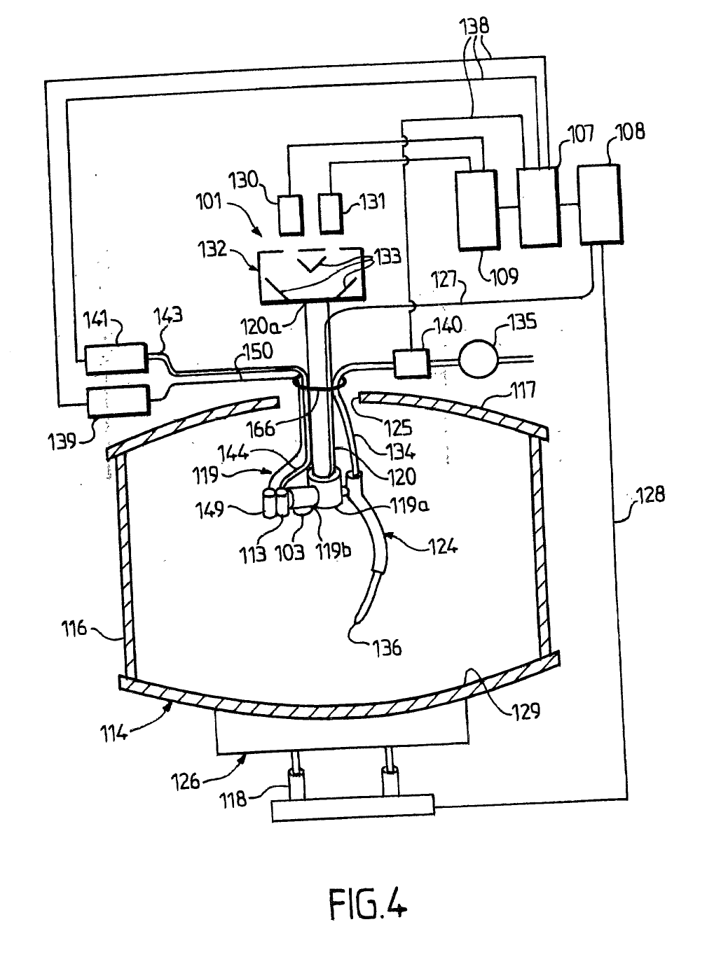

A third embodiment of the device for stripping and scorching a wooden cask is now described with reference to FIGS. 4 to 6, in which elements analogous to those of the above-described embodiments are referred to by the same numerals increased by 100.

In FIG. 4, the wooden cask 114 includes a side wall 117 and two heading pieces 116 and is shown in a lateral sectional view. A bunghole 125 is formed through the top of side wall 117. A rigid waveguide 120 is engaged in cask 114 through bunghole 125. The bunghole 125 may be of a conventional type with a diameter of the order of 50 mm, or with a larger diameter depending on the size of waveguide 120 and the components carried thereon.

Waveguide 120 couples an optical source 101 to an optical head 119. Optical source 101 comprises a pulsed laser source 130 for the stripping treatment of the cask and a continuous laser source 131 for the scorching treatment of the cask. Both laser sources are infrared CO₂ lasers with a central wavelength of 10.6 microns and an overall power between 100 and 200 W. For example, pulsed laser source 130 has a peak power between 30 and 80 MW. An optical coupler 132 is arranged between the outputs of laser sources 130 and 131 and an end 120a of waveguide 120 so as to superimpose the beams produced by both laser sources and feed them into the waveguide 120. Optical coupler 132 comprises mirrors 133 and a cooling system (not shown) for cooling mirrors 133.

Coupled to the other end of waveguide 120 is the optical head 119 of the device, which includes two pivotable portions 119a and 119b. An output lens assembly 103 is fitted to optical head portion 119b. For example, output lens assembly 103 is a convergent lens with diameter 30 mm. In order to aim the laser beams at a given portion of the internal surface of cask 114, optical head portion 119a is capable of modifying the azimuthal angle of output lens assembly 103 and optical head portion 119b is capable of modifying the elevation angle of output lens assembly 103. Optical head portion 119a includes a tilted mirror, not shown, for reflecting the radiation from waveguide 120 to the axial direction of optical head portion 119b. Optical head portion 119b includes a second tilted mirror, not shown, for reflecting the radiation from optical head portion 119a to output lens assembly 103. Output lens assembly 103 allows to configure the laser beams so as to define a cross section of the laser beams.

Scanning actuators, not shown, are provided in optical head portions 119a and 119b for pivoting optical head portions 119a and 119b to aim the laser beams at different portions of internal surface 129 and controlled by a central control unit 107 through robot control interface 108 and control lines 127 so as to scan the entire internal surface 129.

A pipe 124 is secured to optical head 119 and coupled by a flexible coupling 134 to a drawing pump 135. For example, air is sucked up from cask 114 with an average speed of 2 m/s in flexible coupling 134. Pipe 124 is pivoted together with optical head portion 119b with respect to optical head portion 119a. In the embodiment shown, pipe 124 comprises two telescopic portions for adjusting the length of pipe 124. Central control unit 107 controls through robot control interface 108 actuators mounted on pipe 124 for adjusting the length of pipe 124 as a function of the orientation of optical head 119, so as to keep an inlet 136 of pipe 124 close to the portion of internal surface 129 aimed at. In a modified embodiment, not shown, pipe 124 has a fixed length.

The smoke that is generated by the interaction between the radiation and the portion of internal surface 129 is sucked up out of cask 114 through pipe 124, coupling 134 and pump 135 so as to keep a low level of absorbing and/or scattering particles in suspension inside cask 114. An accumulation of smoke inside cask 114 would result in both losing radiation power between optical head 119 and internal surface 129 and sooting up output lens assembly 103, which could lead to overheating and damaging of output lens assembly 103. To further avoid such overheating in operation, a blower (not shown) may be provided for blowing air or an inert gas at output lens assembly 103 so as to cool and clean output lens assembly 103.

As mentioned, central control unit 107 controls actuators for orienting optical head 119 and pipe 124 by way of robot control interface 108. Central control unit 107 also controls laser sources 131 and 130 by way of laser control and synchronization interface 109. Wooden cask 114 lies on a mobile support 126 that is coupled to robot control interface 108 by control line 128. Mobile support 126 comprises hydraulic actuators 118 that allow to raise and lower wooden cask 114.

Before operation, in order to insert optical head 119 into cask 114, mobile support 126 is set to the lowest position. Waveguide 120 and optical head 119 are brought in alignment with bunghole 125. Mobile support 126 is then raised until optical head 119 is appropriately positioned about the center of cask 114.

In a modified embodiment, not shown, adapted for heavier wooden casks, the cask is set in a fixed position on an appropriate support, and waveguide 120 is constructed in the form of a variable length waveguide with an actuator controlled by central control unit 107 for varying the length of waveguide 120. For example, waveguide 120 has two telescopic portions, with a portion having a smaller section able to be extended from and retracted into a portion having a larger section, so as to adjust the overall length of the waveguide.

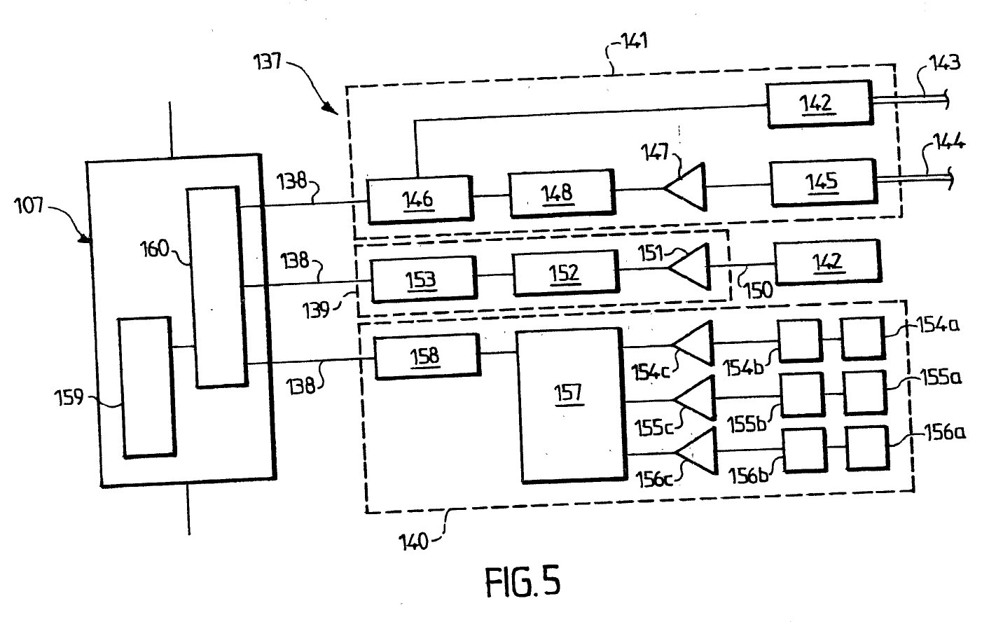

The device of the third embodiment also comprises a measurement system 137, best seen on FIG. 5, coupled to central control unit 107 by measurement data lines 138. Measurement data lines 138 run between central control unit 107 and measurement system 137 and carry control data from central control unit 107 to measurement system 137 and measurement data from measurement system 137 to central control unit 107. The measurement data is processed in real time by central control unit 107 to control and drive the stripping and scorching treatment of the internal surface 129, as will be explained here-below.

With reference to FIGS. 4 and 5, measurement system 137 comprises a calorimetric measurement device for measuring a dominant color of the internal surface under treatment, an acoustic measurement device for measuring ultrasounds generated by the interaction between the laser radiation and the internal surface, and a smoke analyzer 140 for measuring an optical absorption coefficient of the generated smoke.

The calorimetric measurement device comprises a preprocessing unit 141 arranged outside cask 114 and an objective 113 attached to optical head portion 119b inside the cask so as to point to a same portion of internal surface 129 as output lens assembly 103. Preprocessing unit 141 comprises a flashlight 142 coupled to an acquisition trigger module 146 for triggering flashlight 142, and an optical sensor 145 coupled to acquisition trigger module 146 through an amplifier 147 and a color/tension converter 148. An emitting optical fiber 143 runs between an output of flashlight 142 in preprocessing unit 141 and objective 113. A collecting optical fiber 144 runs between objective 113 and an input of optical sensor 145 in preprocessing unit 141. In operation, the calorimetric measurement device operates as a simplified digital camera. When acquisition trigger module 146 triggers an acquisition, a flash of white light is emitted by flashlight 142, carried to objective 113 by fiber 143 and outputted through objective 113 on internal surface 129 whose color is to be measured. The light is reflected by internal surface 129, different spectral components of the flash of light being reflected with different efficiencies depending on the state of internal surface 129. The dominant color of the surface is defined as the visible wavelength or range of wavelength for which the reflectivity of the surface is the highest. The reflected light, including the different spectral components, is collected by objective 113 and carried to optical sensor 145 through fiber 144. Optical sensor 145 includes a number of sensing cells sensitive to different spectral components of the collected light, for example sensing cells for red, sensing cells for green and sensing cells for blue. Optical sensor 145 outputs an electric signal representative of the relative intensity of each of the spectral components of the reflected light. For example, optical sensor 145 is an array of photodiodes or charge-coupled devices (CCD) with spectral filters. Note that a grating spectrometer can also be used as optical sensor 145. The output of optical sensor 145 is amplified by amplifier 147 and converted by converter 148 into an electric signal representative of the dominant color of the reflected light. Acquisition trigger module 146 then supplies color measurement data representative of the dominant color in a format appropriate for processing by central control unit 107.

The acoustic measurement device comprises a preprocessing unit 139 arranged outside cask 114 and an ultrasound directional microphone 149 attached to optical head portion 119b inside the cask so as to be capable of relative orientation with respect to the internal surface, and to point to the portion of internal surface under treatment. Microphone 149 is coupled by a line 150 to an amplifier 151 in preprocessing unit 139. Amplifier 151 is coupled to a filter 152 having a frequency filtering template adapted for filtering out noise and keeping a signal of use. Filter 152 is coupled to an acquisition module 153 for supplying acoustic measurement data representative of the measured ultrasound pattern in a format appropriate for processing by central control unit 107.

Directional microphone 149 can also be replaced by a wide-angle microphone that does not have to be oriented towards a given portion of internal surface 129 to measure the ambient ultrasound in cask 114.

Optical fibers 143 and 144, control line 127, flexible coupling 134 and measurement line 150 are attached to waveguide 120 by an attachment ring 166. They are shielded from waveguide 120 to avoid interference with the laser beams.

Smoke analyzer 140 is arranged between coupling 134 and pump 135 so that the smoke drawn up by pump 135 flows through analyzer 140. Smoke analyzer 140 comprises three pairs of diodes 154a-b, 155a-b and 156a-b arranged on the pathway of the smoke so as to pass a predetermined radiation through a predetermined layer of the smoke between an emitting diode 154a, 155a, 156a of each pair and a receiving diode 154b, 155b, 156b of each pair. Pair of diodes 154a-b passes an infrared light, with wavelength above about 0.8 microns, pair of diodes 155a-b passes a visible light, with wavelength between about 0.4 and 0.8 microns, and pair of diodes 156a-b passes an ultraviolet light with wavelength between about 0.2 and 0.4 microns. Each pair of diodes outputs to a respective amplifier 154c, 155c, 156c a signal representative of the ratio of the radiation power received by receiving diode 154b, 155b and 156b to the radiation power emitted by emitting diode 154a, 155a and 156a, i.e. representative of a transmission coefficient T for each of the three wavelengths or wavelength ranges. For each wavelength or wavelength range, this signal allows to measure an extinction coefficient e of the smoke, which is defined as:

e=−Log(T)/Λ, where Λ is the thickness of the layer of smoke passed through. This extinction coefficient is a function of a chemical composition of the smoke.

A converter module 157 receives the signals outputted by the amplifiers 154c, 155c, 156c and combines them into a frequency modulated electric signal, wherein the respective output of each amplifier 154c, 155c, 156c is converted in a respective band of modulation frequency. A second converter module 158 converts the combined signal outputted by converter module 157 into a measurement signal representative of the extinction coefficient measured for each of the three wavelengths or wavelength ranges in a format suitable for processing by central control unit 107.

Of course, more of less than three pairs of diodes and different values of wavelength can be used for analyzing the smoke.

Smoke analyzer 140 is arranged outside cask 114 because the bulk of smoke analyzer 140 makes it difficult to introduce through bunghole 125. However, in a modified embodiment, not shown, smoke analyzer 140 is constructed in two parts connected together: one part includes the pairs of diodes and is arranged in pipe 124 close to inlet 136, so as to reduce the delay between the time of generation of the smoke and the time of analysis of the smoke, and the other part with a bigger size remains outside cask 114. In all cases, the smoke is drawn up for measuring the optical extinction coefficient of the smoke at a location isolated from the radiation emitted by the portion being stripped or scorched.

Central control unit 107 comprises a comparator unit 160 that is coupled to measurement lines 138 for receiving the measurement signals from the measurement system 137 and a memory unit 159 in which is memorized target data comprising target values for all types of measurement data delivered by measurement system 137. For example, the target data is loaded in memory unit 159 by recording the results of measurements performed with a reference wooden part, or is extracted from a reference database.

Memory unit 159 contains a target color value, for example in the wavelength range between 0.45 and 0.5 micron which corresponds to light oak wood. Comparator unit 160 compares the measured color value received from the calorimetric measurement device to that target color value. In the course of a typical stripping treatment, the dominant color of the internal surface 129 will change from dark red, with wavelength around 0.65 microns, to light oak wood.

Memory unit 159 also contains a target sound pattern, for example in the form of an ultrasound spectrum to be obtained or a dominant wavelength to be obtained. Comparator unit 160 compares the measured sound pattern received from the acoustic measurement device to that target sound pattern.

Memory unit 159 also contains target extinction coefficient values for the three wavelengths used by smoke analyzer 140. Comparator unit 160 compares the measured extinction coefficients received from smoke analyzer 140to those target extinction coefficient values.

The operation of the device in the third embodiment will now be described.

In operation, central control unit 107 controls in real time the movements of optical head 119 and pipe 124 and the operation of laser sources 130 and 131 so as to scan and treat the entire internal surface 129. Since the deposit layer in not homogeneously distributed in the original state of the cask to be treated—for example the layer is thicker on the bottom surface of the cask and absent on the top surface—central control unit 107 also locally adapts the intensity and duration of the stripping and scorching treatment from one portion of the internal surface to the other, as a function of the results of at least one of the above-mentioned comparisons, so as to obtain an homogeneous surface aspect without layer of deposit and with a desired color over the entire internal surface.

Two configurations can be used for outputting both laser beams through lens assembly 103. In a first configuration, the beams are input in waveguide 120 by coupler 132 so as to be coaxial. The radiation generated by both laser sources 130 and 131 is aimed through optical head 119 to precisely the same portion of internal surface 129 for simultaneously carrying out both stripping and scorching treatments of the portion without changing the orientation of optical head 119. This is possible because the pulsed radiation affects the surface layer of deposit while the second radiation affect a layer of wood under the layer of deposit.

In the second configuration, the beams are input in waveguide 120 by coupler 132 so as to be parallel with their respective axes laterally offset, for example by 1 cm. In that configuration, the radiation generated by both laser sources 130 and 131 is aimed through optical head 119 to two respective adjacent portions of internal surface 129. In that case, the stripping and scorching treatments are carried out sequentially for each portion of internal surface 129.

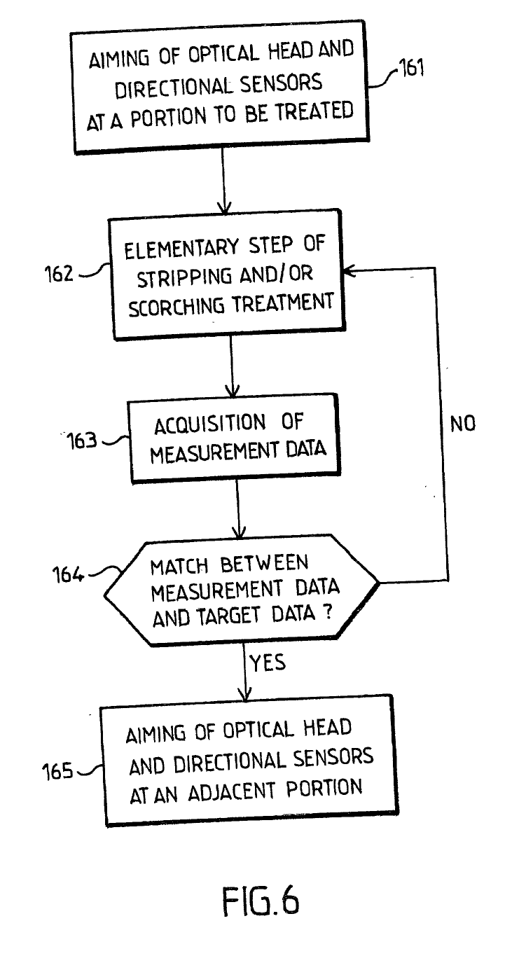

With reference to FIG. 6, the operation of the device comprises the following steps:

At step 161, optical head portion 119b together with output lens assembly 103, microphone 139 and objective 113 is aimed at a given portion of internal surface 129 for the treatment to begin.

At step 162, pulsed laser source 130 and/or continuous laser source 131 is/are actuated to carry out an elementary step of stripping and/or scorching treatment of the portion. For example, the elementary steps consists of a given number of pulses from source 130 and/or a predetermined duration and intensity of scorching irradiation by source 131.

At step 163, while step 162 is carried out, measurement system 137 is actuated to measure at least one of the above-mentioned dominant color, sound pattern and extinction coefficients. In this step, the calorimetric measurement is actually carried out after the given number of laser pulses have been fired or between two pulses.

At step 164, the above-mentioned comparisons between the measurement data acquired at step 163 and the target data are performed. If no substantial match is obtained between the measurement data and the target data, the process of steps 162 and 163 is repeated. For example, the comparisons between the measurement data acquired and the target data indicates that the stripping treatment and/or the scorching treatment of the portion is not sufficient. In that case, step 162 is repeated so as to carry out another elementary step of stripping and/or scorching treatment of the portion, depending on what is needed to obtain the desired final state of surface, i.e. for example the desired color of surface and the desired composition of smoke.

At step 165, when the desired final state of surface is obtained for the portion aimed at, central control unit 107 aims optical head portion 119b together with output lens assembly 103, microphone 139 and objective 113 at an adjacent portion of internal surface 129 for the treatment to continue.

The above steps are repeated until the entire surface is in the desired final state. For example, central control unit 107 is set to operate according to the above-mentioned process by corresponding programming.

Note that instead of constructing optical head 119 with two degrees of rotation, so as to obtain a so-called spherical coordinates scan, the degree of rotation provided by optical head portion 119b, and corresponding to the elevation angle, can be replaced by a degree of translation between the cask 114 and optical head 119 parallel to the axis of waveguide 120, in order to obtain a so-called cylindrical coordinates scan.

Although it is preferred to scan the entire internal surface 129, the top side can be omitted when the layer of deposit is negligible.

### Stripping Step

In the course of the stripping tests which were carried out, it was found that, with a pulsed laser beam having an energy density of 2 J over a surface to be treated of 24 mm², i.e. an energy density of 8 J/cm² which corresponds to a peak power density of 80 MW/cm² for a pulse duration of 100 ns, each pulse causes the removal of 20 μm thickness of wood, which is negligible with respect to the thickness of a stave which is generally between 22 and 27 mm. This is because the coating of organic material is generally intimately linked to a surface layer of the internal surface of the wood, which, during stripping, causes the removal of the organic material and of a layer of wood of a corresponding thickness, which layer is impregnated by the said layer of organic material.

The laser source is preferably a CO₂ laser at atmospheric pressure and operating with transverse excitation (TEA), having a wavelength of 10.6 μm, with a beam output cross section of 16×32 mm.

Preferably, the focusing head will have a long focal length with respect to the distance between the said head and the surface to be treated, so as to reduce the accuracy of positioning the head with respect to the cross section for interaction of the beam with the surface to be treated by producing a beam with a sufficient cross section. Indeed, if the beam were too narrowly focalized on the surface to be treated, it would instantaneously drill a through-hole in the wooden part, which should obviously be avoided. The distance between the optical head 19 and the surface to be treated may vary between 30 and 50 cm.

To industrialize the process, several laser sources, for example three sources, could be coupled in parallel, each one having a pulse frequency of 200 Hz, in order to alternately deliver energy over a same optical path, which makes it possible to obtain an overall pulse frequency of 600 Hz, each pulse having, for example, an energy density of between 150 and 200 mJ/cm², the rate of displacement of the laser beam with respect to the surface to be treated being determined by the central control unit which comprises a computer, so as to obtain an energy density of 8 J/cm² over the surface to be treated.

### Scorching Step

To scorch the wood, three different solutions were tested:

with a CO₂ laser source, for example having a power of 3 kW, and generating an out-of-focus beam having a power density of 180 W/cm²₁ the exposure time needed is greater than 50 ms;

with a CO₂ laser source having a power of 10 W and generating a scanning beam having a power density of about 10 W/cm², the distance between the focusing head and the surface to be treated being about 60 cm, the exposure time needed is markedly longer, about 5 min, in order to obtain the desired scorching, but in this case, the scorching affects a depth of the order of mm and the quantity of energy deposited is much greater;

with an infrared lamp of 1 μm wavelength, having a power of 80 W and a beam aperture angle of 28°, at a distance of about 3 cm from the surface to be treated, the exposure time needed is markedly longer at about 9 min.

These three scorching solutions are not all illustrated in the drawings, but they could be mounted on the robot so as to couple the intense optical source for scorching and the laser source for stripping. A single double-beam optical source could also be provided, producing simultaneously or successively pulsed radiation for stripping and continuous radiation for scorching. These two radiations can be produced simultaneously since their characteristics of interaction with the surface to be treated are markedly different, which avoids any interference.

For the stripping, the number of pulses per unit area, the duration of each pulse and the energy density per unit area, will be able to be determined as a function of the surface state, the quality of the wood and the thickness of the layer to be removed.

For the scorching, the exposure time and the power density of the intense optical source will be determined as a function of the degree of scorching desired by the user.

Although the invention has been described in connection with several particular embodiments, it is clearly obvious that the invention is in no way limited to them and that it comprises all the technical equivalents of the means described as well as their combinations if these come within the scope of the invention.

We claim:

1. A process for stripping and sterilizing the internal surface of a wooden container, having a surface layer of a coating of organic and/or mineral material, comprising:
applying a pulsed radiation produced by an intense infrared optical source over the internal surface, each pulse having a duration and an energy density per unit area to be treated such as to cause sublimation of the said surface layer, the surface of the wood thus stripped being sterilized by the heat released by the radiation.

2. The process according to claim 1, wherein each pulse has a duration of between 10 and 200 ns and an energy of about 2J.

3. Process according to claim 2, wherein each pulse has a duration of about 100 ns and an energy density between 1 and 8 J/cm².

4. The process according to claim 1, characterized in that it comprises applying, over each unit area, from 2 to 20 pulses, preferably between 2 and 10 pulses, depending on the type container to be treated, the state of the surface to be treated and the thickness of the organic and/or mineral coating.

5. The process according to claim 1, wherein the radiation is determined so as to cause a quasiadiabatic sublimation of the layer of organic and/or mineral material on the surface to be treated.

6. The process according to claim 5, wherein 80% of the heat produced by the radiation is absorbed by the surface layer during the sublimation, the remaining 20% being dissipated within the thickness of the wood.

7. The process according to claim 1, wherein each pulse causes the sublimation of about 20 μm thickness of material on the surface to be treated.

8. The process according to claim 1, further comprising evacuating gaseous plasma produced during the sublimation, by sucking up the gaseous plasma or blowing out the gaseous plasma using an inert gas or air.

9. The process according to claim 1, wherein the intense optical source comprises a laser source.

10. The process according to claim 1, further comprising the steps of
starting to strip a portion of the internal surface,
measuring at least one property among a calorimetric property of the portion, an acoustic property of the interaction between the portion and the pulsed radiation and a physical property of smoke generated by the stripping of the portion, and
comparing measurement data corresponding to the said at least one property with predetermined target data representative of a final state of the internal surface to be obtained, and
stopping stripping the portion when the measurement data substantially matches the target data.

11. The process according to claim 10, wherein the step of measuring a calorimetric property of the internal surface portion comprises illuminating the portion with visible light and measuring a spectral property of the light reflected by the portion to determine a dominant color of the portion.

12. The process according to claim 10, wherein the step of measuring a physical property of smoke generated by the stripping of the portion comprises measuring a optical extinction coefficient of the smoke for at least one of an infrared wavelength, a visible wavelength and an ultraviolet wavelength.

13. The process according to claim 10, wherein the step of measuring an acoustic property of the interaction between the portion and the pulsed radiation comprises measuring ultrasounds emitted by a plasma generated by the said interaction.

14. The process according to claim 1, further comprising, simultaneously with or subsequent to the step of stripping and sterilizing, applying over the internal surface of the wooden container a second intense optical radiation, the said second radiation being applied continuously or quasi-continuously for a duration and with an energy density per unit area to be treated to cause scorching of the wood on the surface.

15. The process according to claim 14, wherein the second radiation is applied by a laser source with an out-of-focus beam or by beam scanning.

16. The process according to claim 14, wherein the second radiation has a power density of between 100 and 200 W/cm² for a duration of application of about 0.05 to 0.2 seconds.

17. The process according to claim 16, wherein the second radiation has an energy density per unit area to be treated of about 20 J/cm².

18. The process according to claim 14, wherein the second radiation is applied by an infrared or ultraviolet lamp.

19. The process according to claim 14, further comprising the steps of
starting to scorch a portion of the internal surface, measuring at least one property among a calorimetric property of the portion and a physical property of smoke generated by the scorching of the portion, and
comparing measurement data of the said at least one property with predetermined target data representative of a final state of the internal surface to be obtained, and
stopping scorching the portion when the measurement data substantially matches the target data.

20. The process according to claim 1, wherein each pulse has an energy density of between about 0.5 and 9 J/cm², preferably between 1 and 2 J/cm².

21. A device for stripping and sterilizing the internal surface of a wooden container having a surface layer of a coating of organic and/or mineral material, comprising:
an intense infrared optical source capable of producing pulsed radiation in order to strip and sterilize the said internal surface;
a waveguide connected to an optical output of the source;
an optical focusing head connected to an output of the waveguide, in order to define a cross section of interaction with the internal surface to be treated and thus an energy density to deposit per unit area;
a robot for the relative movement between the optical head and the internal surface of the container to be treated;
a central control unit to control and synthesize source parameters and movements to be carried out by the robot in order to treat the entire internal surface of the container.

22. The device according to claim 21, wherein the source parameters comprise at least one of a number of pulses to be applied per unit area, an impulse frequency and a radiation power of the source.

23. The device according to claim 21, wherein the robot is capable of making the said optical head pivot through an angle of about 120° with respect to the axis of the container, of driving the optical head in relative rotation about the axis of the container with respect to the container, and in driving the container in relative axial translation with respect to the optical head.

24. The device according to claim 21, further comprising a camera for displaying the surface treatment, the camera being connected to a display screen and to the central control unit in order to control the surface treatment visually and in real time.

25. The device according to claim 21, wherein the optical head is arranged so as to penetrate inside the container by way of a bunghole of a cask made of wood, a hole specifically made in a heading piece of a cask made of wood or by one end of a cask of wood from which a heading piece has been removed.

26. The device according to claim 21, further comprising a calorimetric sensor coupled to the central control unit for measuring a dominant color of the internal surface, the calorimetric sensor being capable of relative orientation with respect to the internal surface.

27. The device according to claim 21, further comprising a pipe for sucking up smoke generated by the stripping.

28. The device according to claim 27, further comprising a smoke analyzer coupled to the central control unit for measuring an optical extinction coefficient of the smoke sucked up by the pipe for at least one of an infrared wavelength, a visible wavelength and an ultraviolet wavelength.

29. The device according to claim 27, wherein the robot is capable of moving the pipe in coordination with the optical head such as to keep an inlet portion of the pipe adjacent to a portion of the internal surface at which the optical head is aimed.

30. The device according to claim 21, further comprising a microphone coupled to the central control unit for measuring a sound pattern generated by the interaction between the pulsed radiation and the internal surface.

31. The device according to claim 21, further comprising a second intense optical source for producing a second radiation for scorching the said internal surface.
